(19) United States
(12) Patent Application Publication
(10) Pub. No.: US 2004/0053857 A1
(43) Pub. Date: Mar. 18, 2004

(54) Glycoalkaloid compositions and various uses thereof

(75) Inventors: Stephen John Carter, Woodvale (AU); Elizabeth Williams, Wembley Downs (AU)

Correspondence Address: MERCHANT & GOULD PC, P.O. BOX 2903, MINNEAPOLIS, MN, 55402-0903, US

(73) Assignee: SOLBEC PHARMACEUTICALS LIMITED, Nedlands (AU)

(21) Appl. No.: 10/607,890

(22) Filed: Jun. 27, 2003

Related U.S. Application Data
(60) Provisional application No. 60/393,140, filed on Jul. 1, 2002.

(51) Int. Cl. A61K 31/7052; A61K 31/58

(52) U.S. Cl. 514/26; 514/176

(57) ABSTRACT

A composition comprising at least two glycoalkaloids of formula I:

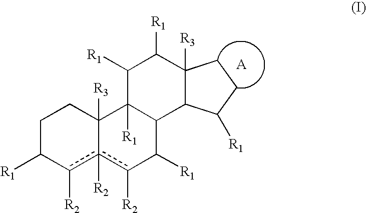

wherein: either one or both of the dotted lines represents a double bond, and the other a single bond, or both represent single bonds;

A: represents a radical selected from the following radicals of general formulae (II) to (V):

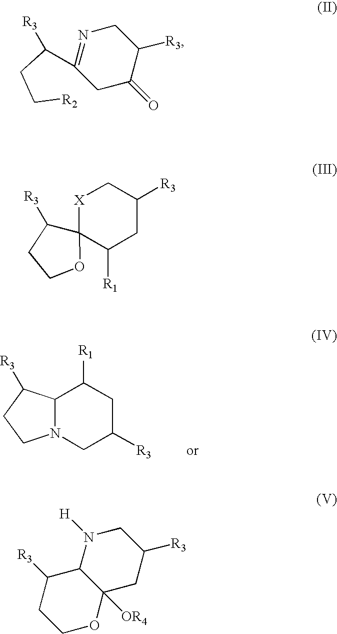

each of R¹ is a radical separately selected from the group consisting of hydrogen, amino, oxo and OR⁴;

each of R² is a radical separately selected from the group consisting of hydrogen, amino and OR⁴;

each of R³ is a radical separately selected from the group consisting of hydrogen, carbohydrate and a carbohydrate derivative;

“X” is a radical selected from the group comprising —CH₂—, —O— and —NH₂—; and

wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates;

wherein the ratio of said glycoalkaloids is between 6:1 and 1:6 and on the proviso that when the glycoalkaloids are solamargine and solasonine and they are present in a 1:1 ratio the solamargine and solasonine are isolated.

The present application claims priority of U.S. Provisional Application No. 60/393,140 filed Jul. 1, 2002.

### FIELD OF THE INVENTION

The present invention relates to compositions including glycoalkaloids such as solasonine and solamargine, to pharmaceutical compositions including glycoalkaloids and to methods of treating various disorders such as cancer and psoriasis with the compositions.

### BACKGROUND ART

BEC® is a therapeutic agent comprising a mixture of the triglycosides: solasonine and solamargine, their corresponding mono and diglycosides, solasodine (an aglycone) and free sugars.

It has previously been recognised that the free sugars in BEC® may reduce its therapeutic activity. However, no studies have been carried out to determine what constituents in BEC® may be used in combination to treat cancer and/or the preferred ratios of any such constituents.

The present invention seeks to overcome or at least partially alleviate this problem.

### SUMMARY OF THE INVENTION

The present invention provides a composition comprising at least two glycoalkaloids of formula I:

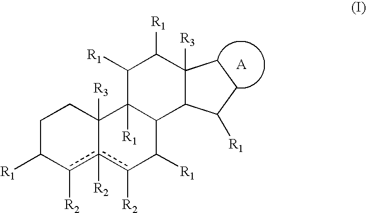

wherein: either one or both of the dotted lines represents a double bond, and the other a single bond, or both represent single bonds;

A: represents a radical selected from the following radicals of general formulae (II) to (V):

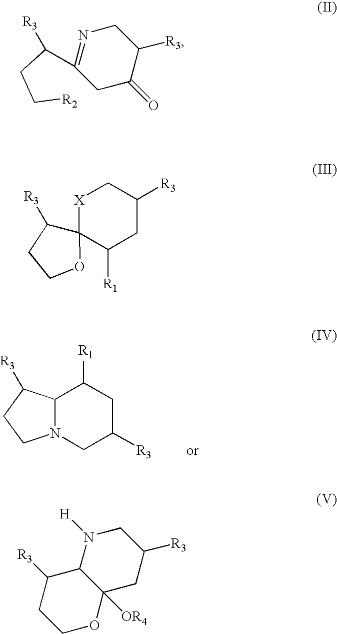

each of R¹ is a radical separately selected from the group consisting of hydrogen, amino, oxo and OR⁴;

each of R² is a radical separately selected from the group consisting of hydrogen, amino and OR⁴;

each of R³ is a radical separately selected from the group consisting of hydrogen, carbohydrate and a carbohydrate derivative;

“X” is a radical selected from the group comprising —CH₂—, —O— and —NH₂—; and

wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates; and

wherein the ratio of said glycoalkaloids is between 6:1 and 1:6 and on the proviso that (i) when the glycoalkaloids are solamargine and solasonine and they are present in a 1:1 ratio the solamargine and solasonine are isolated; or (ii) when the glycoalkaloids are solasonine and solamargine they do not constitute 66% of glycosides in the composition.

The present invention also provides a composition consisting essentially of at least two glycoalkaloids of formula I wherein: either one or both of the dotted lines represents a double bond, and the other a single bond, or both represent single bonds; A: represents a radical selected from the following radicals of general formulae (II) to (V); each of R¹ is a radical separately selected from the group consisting of hydrogen, amino, oxo and OR⁴; each of R² is a radical separately selected from the group consisting of hydrogen, amino and OR⁴; each of R³ is a radical separately selected from the group consisting of hydrogen, carbohydrate and a carbohydrate derivative; “X” is a radical selected from the group comprising —CH₂—, —O— and —NH₂—; and wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates.

The present invention also provides a pharmaceutical composition comprising a glycoalkaloid composition of the present invention and a pharmaceutically acceptable carrier.

The present invention also provides a method of treating cancer comprising the step of administering an effective amount of a composition or pharmaceutical composition of the present invention to a cancer patient.

The present invention also provides a method of treating psoriasis comprising the step of administering an effective amount of a composition or pharmaceutical composition of the present invention to a psoriasis patient.

The present invention also provides a method for contraception comprising the step of administering an effective amount of a composition or pharmaceutical composition of the present invention to a subject to at least reduce and preferably totally remove the subjects ability to fall pregnant.

The present invention also provides a method of terminating a pregnancy comprising the step of administering an effective amount of a composition or pharmaceutical composition of the present invention to a pregnant subject.

The present invention also provides a method of treating a subject infected with a pathogenic organism comprising the step of administering an effective amount of a composition or pharmaceutical composition of the present invention to the infected subject.

The present invention also provides a method of treating abnormal cell growth in a patient comprising the step of administering an effective amount of a composition or pharmaceutical composition of the present invention to a patient with abnormal cell growth.

The present invention also provides a method of diagnosing or identifying abnormal cell growth in a subject comprising the step of applying a composition or pharmaceutical composition of the present invention to a test area on said subject and then monitoring said test area for inflammation.

The present invention also provides a method of diagnosing or identifying abnormal cell growth in a subject comprising the step of administering a composition or pharmaceutical composition of the present invention, incorporating a detectable label, to a subject and then detecting said label.

### BRIEF DESCRIPTION OF THE DRAWINGS

FIGS. 1A-1C depict the results of cell assays assessing the activity of various ratios of solamargine and solasonine in the cell lines NHDF-Ad, 786-0 and N036, respectively.

FIGS. 2A and 2B are photographs depicting a human patient with psoriasis in the elbow region before (2A) and after (2B) treatment with a cream containing 0.1% of a 1:1 ratio of solasonine and solamargine.

### DETAILED DESCRIPTION OF THE INVENTION

Glycoalkaloid compositions

The present invention provides a composition comprising at least two glycoalkaloids of formula I:

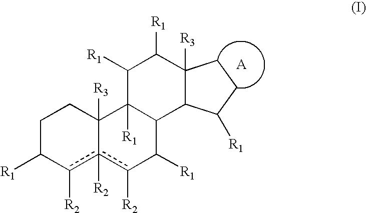

wherein: either one or both of the dotted lines represents a double bond, and the other a single bond, or both represent single bonds;

A: represents a radical selected from the following radicals of general formulae (II) to (V):

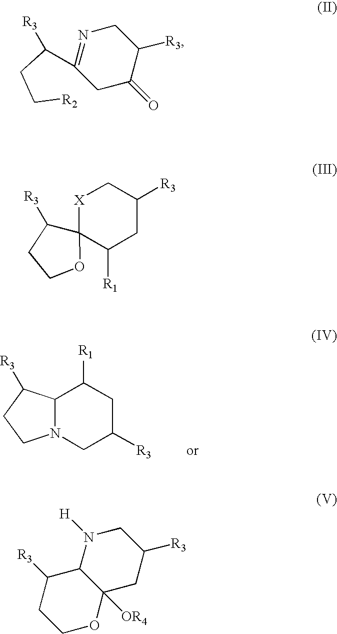

each of R¹ is a radical separately selected from the group consisting of hydrogen, amino, oxo and OR⁴;

each of R² is a radical separately selected from the group consisting of hydrogen, amino and OR⁴;

each of R³ is a radical separately selected from the group consisting of hydrogen, carbohydrate and a carbohydrate derivative;

“X” is a radical selected from the group comprising —CH₂—, —O— and —NH₂—; and

wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates;

wherein the ratio of said glycoalkaloids is between 6:1 and 1:6 and on the proviso that when the glycoalkaloids are solamargine and solasonine and they are present in a 1:1 ratio the solamargine and solasonine are isolated.

Throughout the specification, unless the context requires otherwise, the word “comprise” or variations such as “comprises” or “comprising”, will be understood to imply the inclusion of a stated integer or group of integers but not the exclusion of any other integer or group of integers.

For the purposes of the present invention the term “isolated” means essentially free of (i) mono and diglycosides and, preferably, essentially free of (ii) free sugars such as mono, di, tri, oligo or polysaccharides and (iii) aglycone. However, unless steps are taken to stabilise the glycoalkaloids, it will be appreciated that even in an isolated glycoalkaloid composition of the present invention there will be a small amount of free sugars and mono and diglycosides that result from degradation of the glycoalkaloids.

The number of glycoalkaloids in the composition may be varied, as may their relative ratios in the composition. However, when the composition comprises two glycoalkaloids they may be present in a ratio selected from the group of ratios consisting of approximately: 1:6-1:0.5; 1:5; 1:4; 1:3; 1:2, 1:1.5 and 1:1. In this regard, surprisingly, it has been found that compositions containing particular ratios of glycoalkaloids have a potentiated activity relative to (i) compositions containing the individual glycoalkaloids and (ii) BEC®.

The glycoalkaloids of the present invention may be varied. Preferably, the glycoalkaloids are triglycoside glycoalkaloids, solasodine glycosides or are selected from the group of glycoalkaloids consisting of: solamargine, solasonine, solanine, tomatine, solanocapsine and 26-aminofurostane.

The glycoalkaloids of the present invention may be chiral, steroisomers and mixtures thereof including enantiomers and/or diastereoisomers. Furthermore, the glycoalkaloids of the present invention may be isolated from natural sources, synthesized or produced by chemically modifying other glycoalkaloids.

The present invention also provides a composition comprising solamargine and solasonine in a ratio between about 1:6 and 6:1 on the proviso that when the solamargine and solasonine are present in a 1:1 ratio the solamargine and solasonine are isolated. Preferably, the solamargine:solasonine ratio is between about 1:4 and 4:1, 1:3 and 3:1 or 1:2 to 2:1.

Thus, the present invention also provides a composition comprising about a 1:1 ratio of solamargine and solasonine in isolated form.

The present invention also provides a composition comprising at least two glycoalkaloids of formula I wherein: either one or both of the dotted lines represents a double bond, and the other a single bond, or both represent single bonds; A: represents a radical selected from the following radicals of general formulae (II) to (V); each of R¹ is a radical separately selected from the group consisting of hydrogen, amino, oxo and OR⁴; each of R² is a radical separately selected from the group consisting of hydrogen, amino and OR⁴; each of R³ is a radical separately selected from the group consisting of hydrogen, carbohydrate and a carbohydrate derivative; “X” is a radical selected from the group comprising —CH₂—, —O— and —NH₂—; and wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates; and

on the proviso that when the glycoalkaloids are solasonine and solamargine they do not constitute 66% of glycosides in the composition.

Preferably, the glycoalkaloids are triglycoside alkaloids and constitute greater than 70%-90% of the glycosides in the composition, more preferably 91-95% and even more preferably 96-100% of the glycosides in the composition.

The number of glycoalkaloids in the composition may be varied, as may their relative ratios in the composition. However, when the composition comprises two glycoalkaloids they may be present in a ratio selected from the group of ratios consisting of: 1:5; 1:4; 1:3; 1:2 and 1:1.

Preferably, the glycoalkaloids are selected from the group of glycoalkaloids consisting of: solamargine, solasonine, solanine, tomatine, solanocapsine and 26-aminofurostane.

Particularly preferred glycoalkaloids are solasodine glycosides such as solamargine and solasonine. Thus, the present invention also provides a composition comprising solamargine and solasonine in a ratio between about 1:6 and 6:1, 1:4 and 4:1, 1:3 and 3:1 or 1:2 to 2:1 on the proviso that the solasonine and solamargine do not constitute 66% of the glycosides in the composition.

One particular composition of the present invention comprises about a 1:1 ratio of solamargine and solasonine on the proviso that the solasonine and solamargine do not constitute 66% of the glycosides in the composition.

The present invention also provides a composition consisting essentially of at least two glycoalkaloids of formula I wherein: either one or both of the dotted lines represents a double bond, and the other a single bond, or both represent single bonds; A: represents a radical selected from the following radicals of general formulae (II) to (V); each of R¹ is a radical separately selected from the group consisting of hydrogen, amino, oxo and OR⁴; each of R² is a radical separately selected from the group consisting of hydrogen, amino and OR⁴; each of R³ is a radical separately selected from the group consisting of hydrogen, carbohydrate and a carbohydrate derivative; “X” is a radical selected from the group comprising —CH₂—, —O— and —NH₂—; and wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates.

For the purposes of the present invention the phrase “consisting essentially of” means that the glycoalkaloids in the composition are the only glycosides therein. Thus, a composition consisting essentially of solamargine and solasonine includes solamargine and solasonine and may include other non-glycoside constituents.

Preferably, the glycoalkaloids are triglycoside alkaloids and constitute greater than 70%-90% of the glycosides in the composition, more preferably 91-95% and even more preferably 96-100% of the glycosides in the composition.

The number of glycoalkaloids in the composition may be varied, as may their relative ratios in the composition. However, when the composition consists essentially of two glycoalkaloids they may be present in a ratio selected from the group of ratios consisting of: 1:5; 1:4; 1:3; 1:2 and 1:1.

Preferably, the glycoalkaloids are selected from the group of glycoalkaloids consisting of: solamargine, solasonine, solanine, tomatine, solanocapsine and 26-aminofurostane.

Particularly preferred glycoalkaloids are solasodine glycosides such as solamargine and solasonine. Thus, the present invention also provides a composition consisting essentially of solamargine and solasonine in a ratio between about 1:6 and 6:1, 1:4 and 4:1, 1:3 and 3:1 or 1:2 to 2:1. One particular composition of the present invention consists essentially of about a 1:1 ratio of solamargine and solasonine.

The amount of glycoalkaloids in the compositions of the present invention may be varied depending on their intended end use. Preferably, the compositions comprise about 0.001%-5% or 10% glycoalkaloids, more preferably 0.01%-5% or 10% and even more preferably 0.1%-5% or 10% glycoalkaloids.

Pharmaceutical Compositions

The compositions of the present invention may be capable of administration to a patient to treat a disorder. Thus, the present invention also provides a pharmaceutical composition comprising a glycoalkaloid composition of the present invention and a pharmaceutically acceptable carrier.

Methods for the preparation of pharmaceutical compositions comprising one or more active ingredients are generally known in the art. Such pharmaceutical compositions will generally be formulated for the mode of delivery that is to be used and will usually include one or more pharmaceutically acceptable carriers. A “pharmaceutically acceptable carrier” is a material that is not biologically or otherwise undesirable, i.e., the material can be administered to an individual along with the glycoalkaloid composition without causing unacceptable biological effects or interacting in a deleterious manner with any of the other components of the pharmaceutical composition in which it is contained.

Thus, in addition to the glycoalkaloid composition and aforementioned additives, pharmaceutical compositions of the invention may further comprise suitable carriers, excipient and diluents that are pharmaceutically acceptable and compatible with the active ingredient. Some examples of suitable carriers, excipient and diluents include, without limitation, water, saline, ethanol, dextrose, cyclodextrins such as hydroxy propyl beta-cyclodextrin, glycerol, lactose, dextrose, sucrose sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, talc, magnesium stearate and mineral oil or combinations thereof.

The formulations can additionally include lubricating agents, pH buffering agents, wetting agents, emulsifying and suspending agents, preserving agents, sweetening agents or flavouring agents. The particular selection of constituent that can be included in the compositions described herein will generally depend on the particular mode of delivery used to bring the glycoalkaloid into contact with the target cells or tissue.

Pharmaceutical compositions according to the invention may be administered to a patient using any technology or delivery route that permits contact between the glycoalkaloid and the target cell or tissue. Preferably, the composition is administered by topical or oral means. However, any technology that allows targeted delivery of the pharmaceutical composition via, intravenously, subcutaneously, intramuscularly, intraorbitally, ophthalmically, intraventricularly, intracranially, intracapsularly, intraspinally, intracisternally, intraperitoneally, buccal, rectally, vaginally, intranasally or by pulmonary administration may be used to deliver the composition to a tumorous growth. Suitable dosage forms include for example, pastes, gels, liquids, tablets, troches, dispersions, suspensions, solutions, capsules, patches, suppositories and the like, although oral dosage forms are preferred.

The mode of administration must, however, be at least suitable for the form in which the composition has been prepared. The mode of administration for the most effective response may need to be determined empirically and the means of administration described below are given as examples and do not limit the method of delivery of the composition of the present invention in any way. All the formulations described below are commonly used in the pharmaceutical industry and are commonly known to suitably qualified practitioners.

(a) Topical Delivery

Various topical delivery systems may be appropriate for administering the compositions of the present invention depending upon the preferred treatment regimen. Topical formulations may be produced by dissolving or combining the glycoalkaloid composition of the present invention in an aqueous or nonaqueous carrier. In general, any liquid, cream, or gel, or similar substance that does not appreciably react with the glycoalkaloid or any other of the active ingredients that may be introduced into the composition and which is non-irritating is suitable. Appropriate non-sprayable viscous, semi-solid or solid forms can also be employed that include a carrier compatible with topical application and have a dynamic viscosity preferably greater than water.

Suitable formulations are well known to those skilled in the art and include, but are not limited to, solutions, suspensions, emulsions, creams, gels, ointments, powders, liniments, salves, aerosols, transdermal patches, etc, which are, if desired, sterilized or mixed with auxiliary agents, e.g., preservatives, stabilizers, emulsifiers, wetting agents, fragrances, colouring agents, odour controllers, thickeners such as natural gums etc. Particularly preferred topical formulations include ointments, creams or gels.

Ointments generally are prepared using either (1) an oleaginous base, i.e., one consisting of fixed oils or hydrocarbons, such as white petroleum or mineral oil, or (2) an absorbent base, i.e., one consisting of an anhydrous substance or substances which can absorb water, for example anhydrous lanolin. Customarily, following formation of the base, whether oleaginous or absorbent, the glycoalkaloid is added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfate; hydrophilic colloids, such as acacia colloidal clays, veegum and the like. Upon formation of the emulsion, the glycoalkaloid composition is customarily added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the glycoalkaloid composition is added to the formulation at the desired concentration at a point preceding addition of the gelling agent.

The amount of the glycoalkaloid compound incorporated into a topical formulation is not critical; the concentration should be within a range sufficient to permit ready application of the formulation to the affected tissue area in an amount that will deliver the desired amount of glycoalkaloid to the desired treatment site.

The customary amount of a topical formulation to be applied to an affected tissue will depend upon an affected tissue size and concentration of the glycoalkaloid in the formulation.

(b) Oral Delivery

The pharmaceutical compositions of the present invention may also be adapted for oral administration in such a manner that facilitates delivery of a therapeutically effective concentration of the glycoalkaloid to the target cell or tissue.

The effective dosages of the glycoalkaloid, when administered orally, must take into consideration the diluent, preferably water. The composition preferably contains about 1 to about 200 mg glycoalkaloid. When the compositions are ingested, desirably they are taken on an empty stomach.

Contemplated for use herein are oral solid dosage forms, which are described generally in Martin, Remington's Pharmaceutical Sciences, 18th Ed. (1990 Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatised with various polymers (e.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the formulation will include the glycoalkaloid composition described as part of the invention and inert ingredients that allow for protection against the stomach environment and release of the glycoalkaloid in the intestine.

In some instances it may be desirable that a pharmaceutical composition of the invention release the glycoalkaloid in the stomach, the small intestine (the duodenum, the jejunem, or the ileum), or the large intestine. One skilled in the art has available formulations that will not dissolve in the stomach, yet will release the material in the duodenum or elsewhere in the intestine. Preferably, the release will avoid the deleterious effects of the stomach environment, either by protection of the composition or by release of the glycoalkaloid beyond the stomach environment, such as in the intestine.

To ensure full gastric resistance, a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S and Shellac. These coatings may be used as mixed films.

A coating or mixture of coatings that are not intended for protection against the stomach can also be used on tablets. This can include sugar coatings, or coatings that make the tablet easier to swallow. Capsules may consist of a hard shell (such as gelatine) for delivery of dry therapeutic i.e. powder; for liquid forms, a soft gelatine shell may be used. The shell material of cachets could be thick starch or other edible paper. For pills, lozenges, moulded tablets or tablet triturates, moist massing techniques can be used.

Colourants and flavoring agents may all be included. For example, glycoalkaloid compositions may be formulated (such as by liposome or microsphere encapsulation) and then further contained within an edible product, such as a refrigerated beverage containing colorants and flavouring agents.

One may dilute or increase the volume of the glycoalkaloid composition with an inert material. These diluents could include carbohydrates, especially mannitol, alpha-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the pharmaceutical formulations of the present invention. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite may all be used. Other disintegrants include insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the pharmaceutical composition together to form a hard tablet and include materials from natural products such as acacia, tragacanth, starch and gelatine. Others include methylcellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the glycoalkaloid composition to prevent sticking during the formulation process. Lubricants may be used as a layer between the glycoalkaloid and the die wall and these can include but are not limited to: stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights and Carbowax 4000 and 6000.

Glidants that might improve the flow properties of the composition during formulation and to aid rearrangement during compression might be added. The glidants may include starch, talc, pyrogenic silica and hydrated silicoaluminate.

To aid dissolution of the glycoalkaloid into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the pharmaceutical composition as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the composition either alone or as a mixture in different ratios.

Additives, which potentially enhance uptake of the glycoalkaloids, are for instance the fatty acids oleic acid, linoleic acid and linolenic acid.

Controlled release formulations may be desirable. The compositions could be incorporated into an inert matrix that permits release by either diffusion or leaching mechanisms i.e., gums. Slowly degenerating matrices may also be incorporated into the pharmaceutical composition. Another form of a controlled release is by a method based on the Oros therapeutic system (Alza Corp.), i.e. the composition is enclosed in a semipermeable membrane which allows water to enter and push the composition out through a single small opening due to osmotic effects. Some enteric coatings also have a delayed release effect.

A mix of materials might be used to provide the optimum film coating. Film coating may be carried out in a pan coater or in a fluidised bed or by compression coating. The glycoalkaloid can be included in the pharmaceutical composition as fine multiparticulates in the form of granules or pellets of particle size about 1 mm. The formulation of the glycoalkaloid for capsule administration could also be as a powder, lightly compressed plugs or even as tablets. The glycoalkaloid could be prepared by compression.

Thus, the invention further provides for compositions of microparticles, created from hydrophilic polymers, which contain the glycoalkaloid. The microparticles containing the glycoalkaloid composition may be made by a variety of methods known to those in the art, for example, solvent evaporation, desolvation, complex coacervation, polymer/polymer incompatibility, interfacial polymerisation etc.

In a preferred embodiment of the invention, the hydrophilic polymers forming the microparticles are attached to a targeting protein that acts to enable the microparticle/glycoalkaloid complex to specifically bind selected target cells or tissues bearing the target molecule (e.g. characteristic marker).

In a more preferred embodiment, the hydrophilic polymers are conjugated to the Fab′ fragment of an antibody. Smaller peptides from the hypervariable region or from another peptide interacting with a specific cell surface ligand may also be conjugated to the complexes. It is most preferred that the antibodies or antibody fragments are directed against target molecules associated with cancerous tissues or cells.

It will be appreciated that the targeting protein (e.g. an antibody or an antibody fragment) can be attached to the hydrophilic polymers either before or after formation of the microparticle/glycoalkaloid complex. In a preferred embodiment, the targeting protein is coupled to the hydrophilic polymer, where the targeting protein/hydrophilic polymer is subsequently used to form the microparticle/glycoalkaloid complex. This provides a convenient means for modifying the targeting specificity of an otherwise generic microparticle.

Targeted microparticles may be prepared by incorporating the Fab′ fragment into the microparticles by a variety of techniques well known to those of skill in the art. For example, a biotin conjugated Fab′ may be bound to a microparticle containing a streptavidin. Alternatively, the biotinylated Fab′ may be conjugated to a biotin derivatised microparticle by an avidin or streptavidin linker. Typically about 30 to 125 and more typically about 50 to 100 Fab′ fragments per microparticle/glycoalkaloid complex are used.

The pharmaceutical compositions of the present invention may also be formed into powders or some other form that is suitable for delivery by inhalation. Whilst inhalation may be via the mouth it will be appreciated that the route of delivery may also by via the nose.

These compositions are particularly useful for treatment of diseases or disorders of the respiratory system such as lung cancer or cancer that may affect other parts of the respiratory system. When designing compositions for delivery to the lungs, they are preferably designed to reach the site of the alveoli.

Persons skilled in the art will have all the skills and information necessary to prepare such compositions as a routine exercise. When the compositions are adapted for delivery by inhalation they may contain various doses of active agent and the particular dose will be determined by a skilled practitioner with due consideration to the recipient and the state of the disease to be treated. However, preferably, the compositions contain between about 100 ug-100 mg of glycolalkaloids, about 200 ug-50 mg of glycoalkaloids or 200 ug-10 mg glycoalkaloids.

(c) Parenteral Delivery

The pharmaceutical compositions of the present invention may also be adapted for parenteral administration that facilitates delivery of a therapeutically effective amount of glycoalkaloid to the target cell or tissue.

Pharmaceutical forms suitable for injectable use include sterile aqueous solutions (where water-soluble) or dispersions and sterile powders for the extemporaneous preparation of sterile injectable solutions or dispersion. Alternatively, the glycoalkaloids may be encapsulated in liposomes and delivered in injectable solutions to assist their transport across cell membrane. The solution may be a solvent or dispersion medium containing, for example, water, ethanol, polyol (for example, glycerol, propylene glycol and liquid polyethylene glycol and the like), suitable mixtures thereof and vegetable oils or cyclodextrins such as hydroxy propyl beta-cyclodextrin. Proper fluidity may be maintained, for example, by the use of a coating such as licithin, by the maintenance of the required particle size in the case of dispersion and by the use of superfactants. Prolonged absorption of the injectable compositions can be brought about by the use in the compositions of agents delaying absorption, for example, aluminium monostearate and gelatine.

Sterile injectable solutions may be prepared by incorporating the glycoalkaloids in the required amount in an appropriate solvent with various of the other ingredients enumerated above, as required, followed by filtered sterilisation. Generally, dispersions are prepared by incorporating the various sterilised active ingredient into a sterile vehicle that contains the basic dispersion medium and the required other ingredients from those enumerated above. In the case of sterile powders for the preparation of sterile injectable solutions, the preferred methods of preparation are vacuum drying and freeze-drying techniques that yield a powder of the active ingredient plus, optionally, any additional desired ingredient from previously sterile-filtered solution thereof.

Methods of Treatment/Diagnostics

The present invention also provides a method of treating cancer comprising the step of administering an effective amount of a composition or pharmaceutical composition of the present invention to a cancer patient.

The present invention also provides a method of treating psoriasis comprising the step of administering an effective amount of a composition or pharmaceutical composition of the present invention to a psoriasis patient.

The present invention also provides a method for contraception comprising the step of administering an effective amount of a composition or pharmaceutical composition of the present invention to a subject to at least reduce and preferably totally remove the subjects ability to fall pregnant.

The present invention also provides a method for killing sperm comprising the step of contacting said sperm with an effective amount of a composition or pharmaceutical composition of the present invention.

The present invention also provides a method of terminating a pregnancy comprising the step of administering an effective amount of a composition or pharmaceutical composition of the present invention to a pregnant subject.

The present invention also provides a method of treating a subject infected with a pathogenic organism comprising the step of administering an effective amount of a composition or pharmaceutical composition of the present invention to the infected subject.

The present invention also provides a method of treating or abnormal cell growth in a patient comprising the step of administering an effective amount of a composition or pharmaceutical composition of the present invention to a patient with abnormal cell growth.

The present invention also provides a method of diagnosing abnormal cell growth in a subject comprising the step of applying a composition or pharmaceutical composition of the present invention to a test area on said subject and then monitoring said test area for inflammation.

This diagnostic may be used to diagnose skin cancers such as keratoses, basal cell carcinomas, squamous cell carcinomas and melanomas. Preferably the abnormal cell growth is located on the skin of the subject.

Other diagnostic methods according to the present invention involve the use of a composition of the present invention further comprising a detectable label. In this regard, the ability of the constituents of the compositions of the present invention to bind to and gain entry to target cells (cancer cells and other cells displaying abnormal growth) renders them particularly adapted to be combined with a detectable label that can also become associated with a target cell. Once associated the label can then be detected to arrive at a diagnosis.

The particular choice of label and associated detection system may be varied as required and any one of a number of options would be apparent to those skilled in the art. Examples include radioactive labels/detection systems and the use of labels that are readily visualised under x-ray.

Labels include radio-opaque or other compounds that can be visualised with X-rays, CAT-scans, or MRI. When interpreting the resulting patterns account must be taken on background signal or “noise” from non-cancer cells that also bear the label, albeit at lower levels. However, those skilled in the art are readily able to discern noise from actual signal in performing the diagnosis.

Other labels include fluorescent labels that can be detected with light microscopic, flow cytometric, or fluorometric detection or histologically using immunoelectron microscopy or non-immuno assays, for in situ detection.

In situ detection may be accomplished by removing a histological specimen from a patient, and applying thereto a labelled composition of the present invention. The labelled composition is preferably applied by overlaying it onto a biological sample. Through the use of such a procedure, it is possible to determine not only the presence of the target cells but also their distribution in the examined tissue. Using the present invention, those of ordinary skill will readily perceive that any of a wide variety of histological methods (such as staining procedures) can be modified in order to achieve such in situ detection.

Immunoassays and non-immunoassays using the compositions of the present invention will typically comprise incubating a sample, such as a biological fluid, a tissue extract, freshly harvested cells, or lysates of cells with the labelled composition and detecting the label by any of a number of techniques well-known in the art.

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the labelled compositions. The solid phase support may then be washed with the buffer a second time to remove unbound composition. The amount of bound label on solid support may then be detected by conventional means.

Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to a labelled composition of the present invention. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers or will be able to ascertain the same by use of routine experimentation.

The compositions of the present invention can also be detectably labelled by linking the same to an enzyme. This then renders the composition suitable for use in an enzyme immunoassay (EIA). The enzyme that is bound to the composition will react with an appropriate substrate, preferably a chromogenic substrate, in such a manner as to produce a chemical moiety that can be detected, for example, by spectrophotometric, fluorometric or by visual means. Enzymes which can be used to detectably label the composition include, but are not limited to, malate dehydrogenase, staphylococcal nuclease, delta-5-steroid isomerase, yeast alcohol dehydrogenase, alphaglycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labelling the compositions, it is possible to detect the labelled cells through the use of a radioimmunoassay (RIA). The radioactive isotope can be detected by such means as the use of a gamma counter or a scintillation counter or by autoradiography.

It is also possible to label the compositions with a fluorescent compound. When the fluorescently labelled compositions are exposed to light of the proper wave length, its presence can then be detected due to fluorescence. Among the most commonly used fluorescent labelling compounds are fluorescein isothiocyanate, rhodamine, phycoerythrin, phycocyanin, allophycocyanin, o-phthaldehyde and fluorescamine.

Compositions can also be detectably labelled using fluorescence emitting metals such as ¹⁵²Eu, or others of the lanthanide series. These metals can be attached to the glycoalkaloids in the composition using metal chelating groups such as diethylenetriaminepentacetic acid (DTPA) or ethylenediaminetetraacetic acid (EDTA).

The compositions can also be detectably labelled by coupling the glycoalkaloids therein to a chemiluminescent compound. The presence of the chemiluminescent-tagged entity is then determined by detecting the presence of luminescence that arises during the course of a chemical reaction. Examples of particularly useful chemiluminescent labelling compounds are luminol, isoluminol, theromatic acridinium ester, imidazole, acridinium salt and oxalate ester.

Likewise, a bioluminescent compound may be used to label the compositions of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labelling are luciferin, luciferase and aequorin.

As an alternative to the methods of treatment and diagnosis involving the use of the compositions and pharmaceutical compositions of the present invention that comprise a plurality of glycoalkaloids, it will be appreciated that the methods herein may also comprise administration of the plurality of glycoalkaloids as separate formulations.

When the plurality of glycoalkaloids is administered as separate formulations they are preferably administered in a manner that mimics the delivery of a single formulation containing the glycoalkaloids. Thus, they may be administered simultaneously or in any other fashion that enables the glycoalkaloids to reach the target site simultaneously and achieve their therapeutic effect.

The present invention will now be described with reference to a number of examples. The examples are in no way limiting on the preceding description.

### EXAMPLES

### Example 1

### Relative Activity of Solamargine and Solasonine When Used in Combination

Materials/Methods

Tumour cell lines A2058, NO36 and LS174T and the normal adult dermal fibroblast line NHDF-Ad were used in 5000 cell, six day assays to compare dose per cell at LD₅₀ values for BEC (1:1 solamargine:solasonine), solamargine, solasonine and an equimolar mixture of solamargine and solasonine. Data from the fibroblast cell line NHRE in a 2500 cell 3 day assay was also obtained.

Tumour cell line LNCaP was used at 7500 cells in a six day assay format while 786-O was used in a 650 cell, six day format where dose per cell is not a limiting factor determining LD₅₀ as well as in 5000 cell, three day format where dose per cell does limit LD₅₀.

Results

The results are set out hereunder in Table 1.

TABLE 1
Comparison of in vitro activity

| | | | | |
|---|---|---|---|---|
| | | | | Solamargine and |
| Cell Line | BEC | Solamargine | Solasonine | Solasonine (1:1) |

Dose per cell at LD₅₀, pg/cell of total alkaloid
(Dose per cell limiting)

| | | | | |
|---|---|---|---|---|
| NO36 | 298.0 | 154.8 | 613.2 | 148.4 |
| LNCaP | 428.5 | 121.9 | 432.5 | 117.3 |
| LS174T | 465.2 | 162.8 | >400 | 171.6 |
| A2058 | 247.0 | 199.0 | 685 | 78 |
| 786-O | 321.2 | 208.0 | >400 | 134.4 |
| NHDF-Ad^{A} | 287.8 | 209.2 | 587.6 | 128 |
| NHRE^{A} | 344.7 | 227.9 | 1657.7 | 289.2 |

Dose per cell at LD₅₀, pg/cell of total alkaloid
(Dose per cell not limiting)

| | | | | |
|---|---|---|---|---|
| 786-O | 1354.0 | 1394.0 | 5606.0 | 452.3 |

A - normal cells

Values reported as >400 reflect data sets in which the range of solasonine concentrations used were sub optimal for data fitting to obtain a reliable, precise value of LD₅₀.

### Example 2

### Effect of Solamargine:Solasonine Ratio on Activity

Materials/Methods

Tumour cell lines 786-O and NO36 and normal fibroblasts NHDF-Ad were used in 5000 cell assays, for three, six and six day assays, respectively, to study the effect of solamargine:solasonine ratio on cytotoxicity.

Solutions of 4 mg/mL solamargine and 4 mg/mL solasonine in 3% acetic acid were mixed to provide the various ratios. These mixtures were diluted in the culture medium appropriate to the cell line used to concentrations ranging from 0.32 to 40 ug/mL. Fifty uL aliquots were added to tissue culture wells seeded with 5000 cells in 150 uL culture medium 24 hours previously.

Cell survival was assessed using the MTT cell proliferation assay following four days growth after application of the alkaloids. The formazan absorbance of alkaloid treated wells relative to wells treated with an equivalently dilute acetic acid solution expressed as a percentage has been taken as percentage cell survival.

Results

The results are set out in Table 2 hereunder.

TABLE 2
Effect of solamargine:solasonine ratios on activity

Dose per cell at LD₅₀, (pg)

| Solamargine | | | NHDF-Ad, 6 |
|---|---|---|---|
| (%) | 786-O, 3 day assay | NO36, 6 day assay | day assay |
| 100 | 304 | 209 | 112 |
| 75 | 410 | 161 | 176 |
| 66.7 | 234 | 155 | 96 |
| 50 | 160 | 157 | 128 |
| 33.3 | 236 | 191 | 176 |
| 25 | 334 | 198 | 224 |
| 0 | 500 | 613 | 400 |

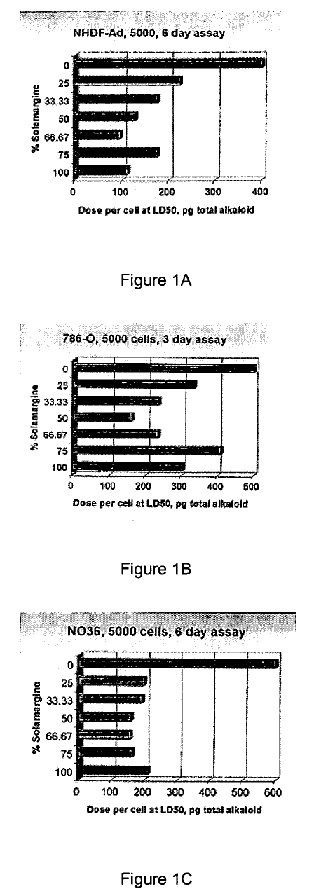

The results in the above table are graphically represented in FIGS. 1A-1C.

### Example 3

### Treatment of Psoriasis

Materials/Methods

A cream containing 0.1% of solasonine and solamargine in a 1:1 ratio was used in this study.

A man with long term and persistent psoriasis was administered a composition of the present invention by applying the above cream to the affected area on a daily basis for three weeks.

Results

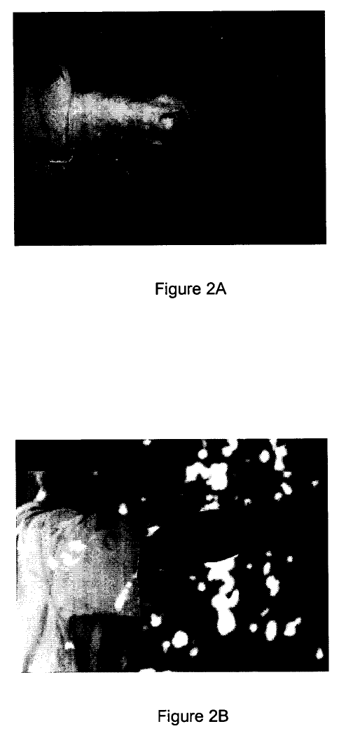

The results are depicted in FIGS. 2A and 2B, which show the affected area of the subject before (2A) and after (2B) treatment with the cream.

The present invention includes modifications and adaptations apparent to one skilled in the art.

Throughout the specification, unless the context requires otherwise, the word “comprise” or variations such as “comprises” or “comprising”, will be understood to imply the inclusion of a stated integer or group of integers but not the exclusion of any other integer or group of integers.

Th claims defining the invention are as follows:

1. A composition comprising at least two glycoalkaloids of formula I: 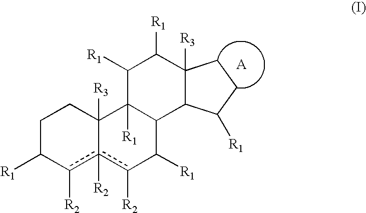
wherein: either one or both of the dotted lines represents a double bond, and the other a single bond, or both represent single bonds;
A: represents a radical selected from the following radicals of general formulae (II) to (V): 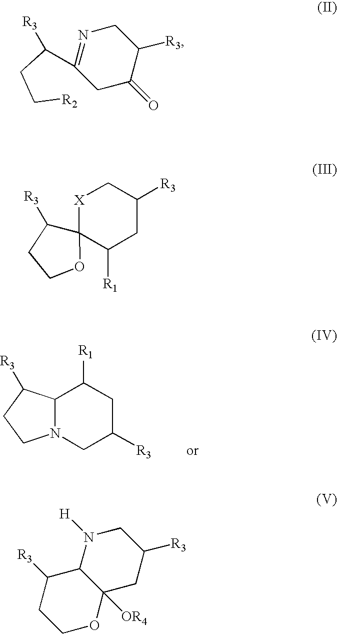
each of R¹ is a radical separately selected from the group consisting of hydrogen, amino, oxo and OR⁴;
each of R² is a radical separately selected from the group consisting of hydrogen, amino and OR⁴;
each of R³ is a radical separately selected from the group consisting of hydrogen, carbohydrate and a carbohydrate derivative;
“X” is a radical selected from the group comprising —CH₂—, —O— and —NH₂—; and
wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates;
wherein the ratio of said glycoalkaloids is between 6:1 and 1:6 and on the proviso that when the glycoalkaloids are solamargine and solasonine and they are present in a 1:1 ratio the solamargine and solasonine are isolated.

2. A composition according to claim 1 comprising two glycoalkaloids in a ratio selected from the group of ratios consisting of approximately: 1:6-1:0.5; 1:5; 1:4; 1:3; 1:2, 1:1.5 and 1:1.

3. A composition according to claim 1 wherein the glycoalkaloids are triglycoside glycoalkaloids or solasodine glycosides.

4. A composition according to claim 1 wherein the glycoalkaloids are selected from the group consisting of: solamargine, solasonine, solanine, tomatine, solanocapsine and 26-aminofurostane.

5. A composition comprising about a 1:1 ratio of solamargine and solasonine in isolated form.

6. A composition comprising at least two glycoalkaloids of formula I wherein:
either one or both of the dotted lines represents a double bond, and the other a single bond, or both represent single bonds;
A: represents a radical selected from the following radicals of general formulae (II) to (V);
each of R¹ is a radical separately selected from the group consisting of hydrogen, amino, oxo and OR⁴; each of R² is a radical separately selected from the group consisting of hydrogen, amino and OR⁴; each of R³ is a radical separately selected from the group consisting of hydrogen, carbohydrate and a carbohydrate derivative; “X” is a radical selected from the group comprising —CH₂—, —O— and —NH₂—; and
wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates; and
on the proviso that when the glycoalkaloids are solasonine and solamargine they do not constitute 66% of glycosides in the composition.

7. A composition according to claim 6 wherein the glycoalkaloids are triglycoside alkaloids and constitute at least 70%-90% of the glycosides in the composition.

8. A composition according to claim 6, wherein the glycoalkaloids are triglycoside alkaloids and constitute at least 91-95% of the glycosides in the composition.

9. A composition according to claim 6, wherein the glycoalkaloids are triglycoside alkaloids and constitute at least 96-100% of the glycosides in the composition.

10. A composition according to claim 6 comprising two glycoalkaloids in a ratio selected from the group of ratios consisting of: 1:5; 1:4; 1:3; 1:2 and 1:1.

11. A composition according to claim 6 wherein the glycoalkaloids are selected from the group of glycoalkaloids consisting of: solamargine, solasonine, solanine, tomatine, solanocapsine and 26-aminofurostane.

12. A composition comprising solamargine and solasonine in a ratio between about 1:6 and 6:1 on the proviso that the solasonine and solamargine do not constitute 66% of the glycosides in the composition.

13. A composition comprising solamargine and solasonine in a ratio between about 1:4 and 4:1 on the proviso that the solasonine and solamargine do not constitute 66% of the glycosides in the composition.

14. A composition comprising solamargine and solasonine in a ratio between about 1:3 and 3:1 on the proviso that the solasonine and solamargine do not constitute 66% of the glycosides in the composition.

15. A composition comprising solamargine and solasonine in a ratio between about 1:2 and 2:1 on the proviso that the solasonine and solamargine do not constitute 66% of the glycosides in the composition.

16. A composition comprising a 1:1 ratio of solamargine and solasonine on the proviso that the solasonine and solamargine do not constitute 66% of the glycosides in the composition.

17. A composition consisting essentially of at least two glycoalkaloids of formula I wherein:
either one or both of the dotted lines represents a double bond, and the other a single bond, or both represent single bonds;
A: represents a radical selected from the following radicals of general formulae (II) to (V); each of R¹ is a radical separately selected from the group consisting of hydrogen, amino, oxo and OR⁴; each of R² is a radical separately selected from the group consisting of hydrogen, amino and OR⁴; each of R³ is a radical separately selected from the group consisting of hydrogen, carbohydrate and a carbohydrate derivative; “X” is a radical selected from the group comprising —CH₂—, —O— and —NH₂—; and
wherein the compound includes at least one R⁴ group that is a carbohydrate or a derivative thereof selected from the group comprising glyceric aldehyde, glycerose, erythrose, threose, ribose, arabinose, xylose, lyxose, altrose, allose, gulose, mannose, glucose, idose, galactose, talose, rhamnose, dihydroxyactone, erythrulose, ribulose, xylulose, psicose, fructose, sorbose, tagatose, and other hexoses, heptoses, octoses, nanoses, decoses, deoxysugars with branched chains, (e.g. apiose, hamamelose, streptose, cordycepose, mycarose and cladinose), compounds wherein the aldehyde, ketone or hydroxyl groups have been substituted (e.g. N-acetyl, acetyl, methyl, replacement of CH₂OH), sugar alcohols, sugar acids, benzimidazoles, the enol salts of the carbohydrates, saccharinic acids, sugar phosphates.

18. A composition according to claim 17 wherein the glycoalkaloids are triglycoside alkaloids and constitute at least 70%-90% of the glycosides in the composition.

19. A composition according to claim 17 wherein the glycoalkaloids are triglycoside alkaloids and constitute at least 91-95% of the glycosides in the composition.

20. A composition according to claim 17 wherein the glycoalkaloids are triglycoside alkaloids and constitute at least 96-100% of the glycosides in the composition.

21. A composition according to claim 17 wherein the composition consists essentially of two glycoalkaloids in a ratio selected from the group of ratios consisting of: 1:5; 1:4; 1:3; 1:2 and 1:1.

22. A composition according to claim 17 wherein the glycoalkaloids are selected from the group of glycoalkaloids consisting of: solamargine, solasonine, solanine, tomatine, solanocapsine and 26-aminofurostane.

23. A composition consisting essentially of solamargine and solasonine in a ratio between about 1:6 and 6:1, 1:4 and 4:1, 1:3 and 3:1 or 1:2 to 2:1.

24. A composition consisting essentially of about a 1:1 ratio of solamargine and solasonine.

25. A composition according to claim 1, 6 or 17 comprising about 0.001%-5% glycoalkaloids.

26. A composition according to claim 1, 6 or 17 comprising about 10% glycoalkaloids.

27. A pharmaceutical composition comprising a composition of claim 1, 6 or 17 and a pharmaceutically acceptable carrier.

28. A pharmaceutical composition according to claim 27 adapted for topical delivery.

29. A pharmaceutical composition according to claim 27 adapted for oral delivery.

30. A pharmaceutical composition according to claim 27 adapted for parenteral delivery.

31. A method of treating cancer in a subject comprising the step of administering to the subject an effective amount of a composition of claim 1, 6 or 17 or a pharmaceutical composition of claim 27.

32. A method of treating psoriasis in a subject comprising the step of administering to the subject an effective amount of a composition of claim 1, 6 or 17 or pharmaceutical composition of claim 27.

33. A method of treating or abnormal cell growth in a patient comprising the step of administering an effective amount of a composition of claim 1, 6 or 17 or pharmaceutical composition of claim 27 to the patient.

34. A method of diagnosing abnormal cell growth in a subject comprising the step of applying a composition of claim 1, 6 or 17 or pharmaceutical composition of claim 27 to a test area on said subject and then monitoring said test area for inflammation.

35. A method according to claim 34 wherein the diagnostic is for skin cancers such as keratoses, basal cell carcinomas, squamous cell carcinomas and melanomas.

36. A composition of claim 1, 6 or 17 further comprising a detectable label.

37. Use of a composition according to claim 36 for detecting a target cell comprising the steps of applying the composition to a sample or a subject and detecting the label.
